(19) United States
(12) Patent Application Publication
(10) Pub. No.: US 2025/0115855 A1
(43) Pub. Date: Apr. 10, 2025

(54) INCUBATOR

(71) Applicants: CANON MEDICAL SYSTEMS CORPORATION, Tochigi (JP); Canon Kabushiki Kaisha, Tokyo (JP)

(72) Inventors: Yosuke YAMAGUCHI, Kawasaki (JP); Noritaka UKIYO, Tokyo (JP)

(73) Assignees: CANON MEDICAL SYSTEMS CORPORATION, Tochigi (JP); Canon Kabushiki Kaisha, Tokyo (JP)

(21) Appl. No.: 18/906,457

(22) Filed: Oct. 4, 2024

(30) Foreign Application Priority Data
Oct. 6, 2023 (JP) 2023-174550

(51) Int. Cl. C12M 1/00; C12M 1/12

(52) U.S. Cl. CPC C12M 41/14; C12M 25/02

(57) ABSTRACT

An incubator according to the present embodiment includes: an incubator main body, a generating unit, a first adjusting unit, and a second adjusting unit. The incubator main body is configured to culture cells at a first temperature and with a first humidity. The generating unit is configured to generate vapor by heating water. The first adjusting unit is provided between the generating unit and the incubator main body and is configured to decrease temperature of the vapor to a second temperature lower than the first temperature and to cause the temperature-decreased vapor to flow into the incubator main body. The second adjusting unit is configured to adjust temperature inside the incubator main body.

### CROSS-REFERENCE TO RELATED APPLICATIONS

This application is based upon and claims the benefit of priority from Japanese Patent Application No. 2023-174550, filed on Oct. 6, 2023; the entire contents of which are incorporated herein by reference.

### FIELD

Embodiments described herein relate generally to an incubator.

### BACKGROUND

Cells may be cultured in an incubator realized with a constant temperature reservoir. In that situation, the cells are cultured in the incubator at a desired temperature and with a desired humidity. On such occasion, to avoid condensation in the incubator, it is necessary to set the temperature and the humidity in the incubator to be a desired temperature and a desired humidity.

### BRIEF DESCRIPTION OF THE DRAWINGS

FIG. 1 is a block diagram illustrating a configuration of an incubator according to an embodiment of the present disclosure; and

FIG. 2 is a drawing for explaining controlling temperature and humidity of the incubator according to the present embodiment.

### DETAILED DESCRIPTION

An incubator according to an embodiment of the present disclosure includes: an incubator main body, a generating unit, a first adjusting unit, and a second adjusting unit. The incubator main body is configured to culture cells at a first temperature and with a first humidity. The generating unit is configured to generate vapor by heating water. The first adjusting unit is provided between the generating unit and the incubator main body and is configured to decrease temperature of the vapor to a second temperature lower than the first temperature and to cause the temperature-decreased vapor to flow into the incubator main body. The second adjusting unit is configured to adjust temperature inside the incubator main body.

Exemplary embodiments of an incubator will be explained in detail below, with reference to the accompanying drawings. Possible embodiments are not limited to the embodiments described below. Further, the description of any of the embodiments is, in principle, similarly applicable to any other embodiment.

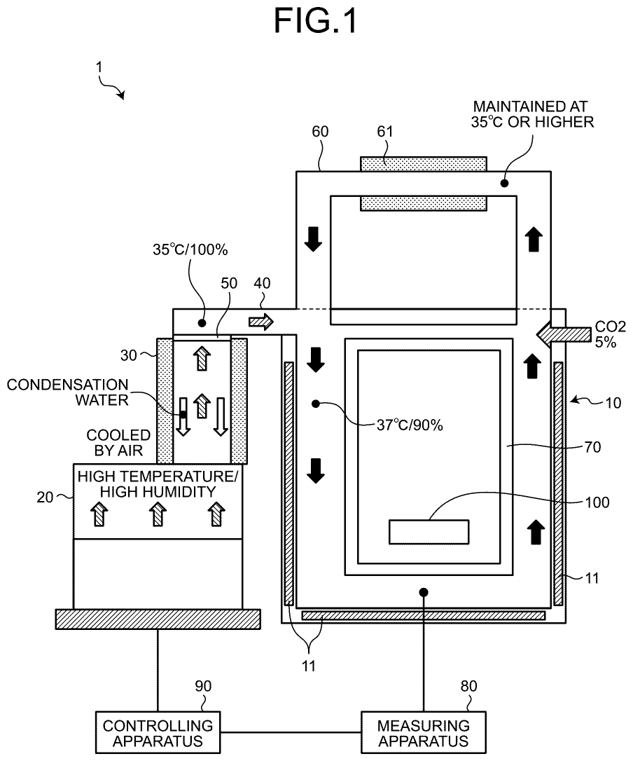

FIG. 1 is a block diagram illustrating a configuration of an incubator 1 according to an embodiment of the present disclosure.

As illustrated in FIG. 1, the incubator 1 according to the present embodiment includes an incubator main body 10.

A culturing unit 100 is accommodated in the incubator main body 10. The culturing unit 100 may be one selected from among: a culturing container; a culturing apparatus holding the culturing container; and a cartridge accommodating the culturing apparatus. The incubator main body 10 is a constant temperature reservoir used for culturing cells at temperature T1 and with humidity H1 and is configured to circulate air inside the incubator main body 10 and carbon dioxide (CO2, 5%) caused to flow in from the outside.

For example, temperature T1 and humidity H1 may be 37° C. and a relative humidity of 90%, respectively. Temperature T1 and humidity H1 are examples of the “first temperature” and the “first humidity”, respectively.

The culturing unit 100 is provided in a casing 70 having a mesh attached. Because of the mesh of the casing 70, it is possible to realize sufficient ventilation (CO2 gas exchange) for the cell culturing performed by the culturing unit 100.

In relation to the above, to prevent condensation in the incubator main body 10, it is necessary to control temperature and humidity of the air circulated in the incubator main body 10. In particular, when the incubator is miniaturized, it is necessary to more strictly control the temperature and the humidity of the air circulated in the incubator main body 10.

For this purpose, the incubator 1 according to the present embodiment includes: the incubator main body 10 used for culturing the cells at a first temperature and with a first humidity; a generating unit (a heated humidifier 20) configured to generate vapor by heating water; a first adjusting unit; and a second adjusting unit. The first adjusting unit is provided between the generating unit and the incubator main body 10 and is configured to decrease the temperature of the vapor to a second temperature lower than the first temperature and to cause the temperature-decreased vapor to flow into the incubator main body 10. The second adjusting unit is connected to the incubator main body 10 and is configured to adjust the temperature inside the incubator main body 10. In this situation, the second temperature may be a temperature at which a humidity amount equivalent to the first temperature and the first humidity turn out to be equal to the amount of saturated water vapor. Further, the second adjusting unit may be configured to adjust the temperature inside the incubator main body 10 to be within the range from the second temperature to the first temperature.

Next, the heated humidifier 20, the first adjusting unit, and the second adjusting unit of the incubator 1 according to the present embodiment will be explained.

As illustrated in FIG. 1, the incubator 1 according to the present embodiment further includes the heated humidifier 20, a cooling tube 30, and a pipe 40.

The heated humidifier 20 is configured to generate the vapor by heating water. The heated humidifier 20 is an example of the “generating unit”. The pipe 40 is provided between the heated humidifier 20 and the incubator main body 10.

The cooling tube 30 is provided between the heated humidifier 20 and the incubator main body 10. Examples of the cooling tube 30 include a stainless spiral cooling tube. The cooling tube 30 is an example of the “first adjusting unit”.

The cooling tube 30 is configured to adjust the temperature of the vapor generated by the heated humidifier 20 to be temperature T0 at which a humidity amount equivalent to temperature T1 and humidity H1 turn out to be equal to the amount of saturated water vapor (a relative humidity of 100%) For example, temperature T0 may be 35° C. Temperature TO is an example of the “second temperature”.

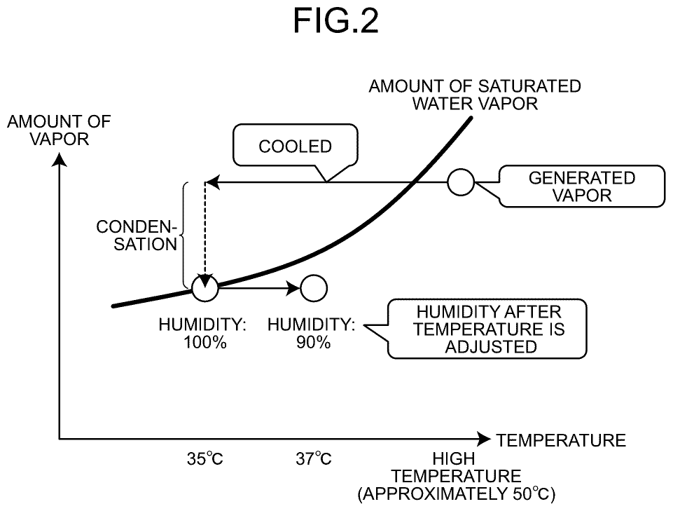

FIG. 2 is a drawing for explaining controlling temperature and humidity of the incubator 1 according to the present embodiment.

As illustrated in FIG. 2, when the temperature increases, the amount of saturated water vapor becomes larger. In this situation, when the temperature of the vapor generated by the heated humidifier 20 being as high as approximately 50° C. is cooled down to 35° C., condensation occurs due to a difference in the amounts of vapor. In that situation, the cooling tube 30 is configured to adjust the temperature of the vapor generated by the heated humidifier 20 to be temperature T0 (35° C.) at which a humidity amount equivalent to temperature T1 (37° C.) and humidity H1 (a relative humidity of 90%) turn out to be equal to the amount of saturated water vapor (a relative humidity of 100%).

The pipe 40 is provided between the cooling tube 30 and the incubator main body 10. The pipe 40 is configured to cause the vapor from the cooling tube 30 to flow into the incubator main body 10.

As illustrated in FIG. 1, the incubator 1 according to the present embodiment further includes a heater 11.

The heater 11 is provided for the incubator main body 10. The heater 11 is configured to heat the vapor having been provided from the pipe 40 and being at temperature T0 (35° C.) and corresponding to the amount of saturated water vapor (a relative humidity of 100%), so as to maintain the temperature and the humidity inside the incubator main body 10 at temperature T1 (37° C.) and with humidity H1 (a relative humidity of 90%). In other words, the heater 11 is configured to maintain the surroundings of the casing 70 in which the culturing unit 100 is provided to be at temperature T1 (37° C.) and with humidity H1 (a relative humidity of 90%).

As illustrated in FIG. 1, the incubator 1 according to the present embodiment further includes a duct 60 and a heat dissipating unit 61. The heater 11, the duct 60, and the heat dissipating unit 61 are examples of the “second adjusting unit”.

The duct 60 is connected to the incubator main body 10 and is provided with a fan (not illustrated). For example, the heat dissipating unit 61 is structured by using a heat dissipation fin and is configured to discharge heat inside the duct 60 to the outside of the incubator 1. There may be situations in which the temperature inside the incubator main body 10 is 37.5° C., for example, being higher than temperature T1 (37° C.). To cope with those situations, the air inside the incubator main body 10 which has flowed into the duct 60 due to ventilation by the fan is cooled by the heat dissipating unit 61 and returned to the inside of the incubator main body 10, so that the temperature inside the incubator main body 10 is adjusted to be within the range from temperature T0 (35° C.) to temperature T1 (37° C.). In contrast, the heater 11 heats the air inside the incubator main body 10 so that the temperature inside the incubator main body 10 does not become lower than temperature T0 (35° C.).

As explained above, in the incubator 1 according to the present embodiment, to avoid condensation within the incubator main body 10, the temperature inside the incubator main body 10 is adjusted so as to be within the range from temperature T0 (35° C.) to temperature T1 (37° C.).

In this situation, as explained above, the cooling tube 30 is configured to adjust the temperature of the vapor generated by the heated humidifier 20 to be temperature T0 at which the humidity amount equivalent to temperature T1 and humidity H1 turn out to be equal to the relative humidity of 100%. In that situation, at the time of cooling the temperature of the vapor, condensation would occur in the cooling tube 30.

To cope with this situation, as illustrated in FIG. 1, the incubator 1 according to the present embodiment further includes a hydrophobic filter 50. The hydrophobic filter 50 is an example of the “filter”.

The hydrophobic filter 50 is provided between the cooling tube 30 and the pipe 40. For example, the hydrophobic filter 50 is provided at an exit of the cooling tube 30 and is configured to cause condensation water to drip into the heated humidifier 20.

The pipe 40 is provided between the hydrophobic filter 50 and the incubator main body 10. The pipe 40 is configured to cause the vapor of temperature T0 adjusted by the cooling tube 30 to flow into the incubator main body 10.

As explained above, the incubator 1 according to the present embodiment is configured so that, before the vapor flows into the incubator main body 10, the condensation water which may be a cause of condensation in the incubator main body 10 is caused by the hydrophobic filter 50 to drip.

Although, in the present embodiment, the hydrophobic filter 50 is presented as an example of the filter used for causing the condensation water to drip, possible embodiments are not limited to this example. For instance, it is acceptable to use any element that is meshed and capable of causing the condensation water to drip.

In relation to the above, there may be some situations in which the humidity inside the incubator main body 10 is not humidity H1 (a relative humidity of 90%), although the temperature inside the incubator main body 10 is temperature T1 (37° C.).

To cope with those situations, as illustrated in FIG. 1, the incubator 1 according to the present embodiment further includes a measuring apparatus 80 and a controlling apparatus 90.

The measuring apparatus 80 is configured to measure the humidity inside the incubator main body 10. The measuring apparatus 80 is an example of the “measuring unit”.

The controlling apparatus 90 is configured to change temperature T0 in accordance with the humidity measured by the measuring apparatus 80. For example, when humidity H1 inside the incubator main body 10 has been changed, the controlling apparatus 90 is configured to control the heated humidifier 20, for example, so as to change temperature T0 in accordance with the humidity inside the incubator main body 10 measured by the measuring apparatus 80. For example, the controlling apparatus 90 may be configured to change temperature T0 adjusted by the cooling tube 30, by changing the temperature (approximately 50° C.) of the vapor generated by the heated humidifier 20 in accordance with the humidity inside the incubator main body 10 measured by the measuring apparatus 80. The controlling apparatus 90 is an example of the “controlling unit”.

In this manner, the incubator 1 according to the present embodiment is configured so that temperature T0 is changed in accordance with the humidity inside the incubator main body 10. Thus, the incubator main body 10 is able to culture the cells at temperature T1 and with humidity H1 at all times.

As explained above, in the incubator 1 according to the present embodiment, the generating unit (the heated humidifier 20) is configured to generate the vapor by heating water. Further, the first adjusting unit (the cooling tube 30) provided between the generating unit and the incubator main body 10 is configured to decrease the temperature of the vapor to the second temperature lower than the first temperature and to cause the temperature-decreased vapor to flow into the incubator main body 10. The second adjusting unit (the duct 60 and the heat dissipating unit 61) connected to the incubator main body 10 is configured to adjust the temperature inside the incubator main body 10. As a result, in the incubator 1 according to the present embodiment, condensation does not easily occur inside the incubator main body 10. In particular, it is possible to carry out the humidity control more precisely, by ensuring that the second temperature is a temperature at which the humidity amount equivalent to the first temperature and the first humidity turn out to be equal to the amount of saturated water vapor. In addition, because the second adjusting unit (the duct 60 and the heat dissipating unit 61) is configured to adjust the temperature inside the incubator main body 10 to be within the range from the second temperature to the first temperature, it is possible to prevent condensation and to keep the inside of the incubator main body 10 with an appropriate level of humidity.

According to at least one aspect of the embodiments described above, it is possible to provide an incubator on the inside of which condensation does not easily occur.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An incubator comprising:
an incubator main body used for culturing cells at a first temperature and with a first humidity;
a generating unit configured to generate vapor by heating water;
a first adjusting unit provided between the generating unit and the incubator main body and configured to decrease temperature of the vapor to a second temperature lower than the first temperature and to cause the temperature-decreased vapor to flow into the incubator main body; and
a second adjusting unit configured to adjust temperature inside the incubator main body.

2. The incubator according to claim 1, wherein the second temperature is a temperature at which a humidity amount equivalent to the first temperature and the first humidity turn out to be equal to an amount of saturated water vapor.

3. The incubator according to claim 2, wherein
the first temperature is 37° C.,
the first humidity is a relative humidity of 90%, and
the second temperature is 35° C. at which a humidity amount equivalent to 37° C. and the relative humidity of 90% turn out to be a relative humidity of 100%.

4. The incubator according to claim 1, wherein the second adjusting unit is configured to adjust the temperature inside the incubator main body to be within a range from the second temperature to the first temperature.

5. The incubator according to claim 1, further comprising: a filter provided between the first adjusting unit and the incubator main body and used for causing condensation water to drip.

6. The incubator according to claim 5, wherein the filter is a hydrophobic filter.

7. The incubator according to claim 1, further comprising:
a measuring unit configured to measure humidity of the incubator main body; and
a controlling unit configured to change the second temperature in accordance with the humidity measured by the measuring unit.

8. The incubator according to claim 1, further comprising: a controlling unit configured to change the second temperature when the first humidity has been changed.
